(19) United States
(12) Patent Application Publication
(10) Pub. No.: US 2023/0190703 A1
(43) Pub. Date: Jun. 22, 2023

(54) TREATING SAR-COV-2 RELATED HEALTH PROBLEMS

(71) Applicant: Allen Qian, Manhasset, NY (US)

(72) Inventor: Allen Qian, Manhasset, NY (US)

(21) Appl. No.: 17/555,736

(22) Filed: Dec. 20, 2021

(51) Int. Cl. A61K 31/366; A61P 31/14; A61K 31/357

(52) U.S. Cl. CPC A61K 31/366; A61P 31/14; A61K 31/357

(57) ABSTRACT

A method of treating Sar-cov-2 re-infection in a subject, a method of reducing side effects in a subject suffering from side effects of Covid-19 vaccination, a method of reducing apoptosis of immune cells or gut cells caused by a Sar-cov-2 spike protein in a subject, a method of reducing necrosis of immune cells or gut cells caused by a Sar-cov-2 spike protein in a subject, a method of increasing CD26 level in immune cells in a subject infected with Sar-cov-2, and a method of decreasing cell adhesion molecule level in immune cells in a subject infected with Sar-cov-2 are disclosed. Each method comprises administering an effective amount of a pharmaceutical composition comprising artemisinin or artesunate to said subject. A method of making an anti-Sar-cov-2 compound by making and testing derivatives artemisinin or artesunate is disclosed.

### FIELD

This disclosure relates to Immunology and Infectious Disease.

### BACKGROUND

The Sars-Cov-2 virus causes Covid-19 in humans. Although vaccines exist and some treatments as well, the Covid-19 pandemic continues to be a major health problem.

### SUMMARY

In one aspect, this disclosure provides a method of treating Sar-cov-2 re-infection in a subject, wherein said re-infection is caused by a Sar-cov-2 virus variant that has bypassed the subject's humoral immune system, comprising administering a therapeutically effective amount of a pharmaceutical composition comprising artemisinin or artesunate to said subject.

In another aspect, this disclosure provides a method of reducing side effects in a subject suffering from side effects of Covid-19 vaccination, comprising administering an effective amount of a pharmaceutical composition comprising artemisinin or artesunate to said subject.

In another aspect, this disclosure provides a method of reducing apoptosis of immune cells or gut cells caused by a Sar-cov-2 spike protein in a subject, comprising administering an effective amount of a pharmaceutical composition comprising artemisinin or artesunate to said subject.

In yet another aspect, this disclosure provides a method of reducing necrosis of immune cells or gut cells caused by a Sar-cov-2 spike protein in a subject, comprising administering an effective amount of a pharmaceutical composition comprising artemisinin or artesunate to said subject.

In another aspect, this disclosure provides a method of increasing CD26 level in immune cells in a subject infected with Sar-cov-2, comprising administering an effective amount of a pharmaceutical composition comprising artemisinin or artesunate to said subject.

In another aspect, this disclosure provides a method of decreasing cell adhesion molecule level in immune cells in a subject infected with Sar-cov-2, comprising administering an effective amount of a pharmaceutical composition comprising artemisinin or artesunate to said subject.

In yet another aspect, this disclosure provides a method of making an anti-Sar-cov-2 compound comprising making and testing derivatives of artemisinin or artesunate for activity of one or more of: increasing CD26 level in immune cells with lowered CD26 level caused by Sar-cov-2 spike protein, increasing CD26 level in immune cells, and reducing apoptosis or necrosis of immune cells or gut cells caused by a Sar-cov-2 spike protein.

### BRIEF DESCRIPTION OF THE DRAWINGS

The accompanying drawings, which are incorporated into and constitute a part of this specification, illustrate one or more embodiments of the present disclosure and, together with the detailed description and the examples, serve to explain the principles and implementations of the disclosure.

FIG. 1 shows effect of SPK (Sar-cov-2 spike protein), A1 (Artemisinin), and A2 (Artesunate) on cell adhesion. See Example 1. (p<0.05*).

FIG. 2 shows effect of SPK, A1 and A2 on percent cytotoxicity. See Example 1. (p<0.05*).

FIG. 3 shows effect of SPK, A1 and A2 on caspase activity. See Example 1. (p<0.05*).

FIG. 4 shows effect of SPK on colo 320 cell caspase activity. (p<0.05*). See Example 2. The spike protein (SPK) is referred to as SP. (p<0.05*).

FIG. 5 shows effect of SPK on colo 320 cells, percent cytotoxicity. (p<0.05*). See Example 2. The spike protein (SPK) is referred to as SP.

FIG. 6 shows effect of A1 or A2 and SPK on percent apoptosis. (p<0.05*). See Example 2. The spike protein (SPK) is referred to as SP.

FIG. 7 shows effect of A1 or A2 and SPK on percent cytotoxicity. (p<0.05*). See Example 2. The spike protein (SPK) is referred to as SP.

### DETAILED DESCRIPTION

As used herein, the word “a” or “plurality” before a noun represents one or more of the particular noun.

The word “comprise” or variations such as “comprises” or “comprising” will be understood to imply the inclusion of a stated integer or groups of integers but not the exclusion of any other integer or group of integers.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. Any one or more individual members of a range or group disclosed herein may be excluded from a claim of this disclosure. The disclosure illustratively described herein suitably may be practiced in the absence of any element or elements, limitation or limitations which is not specifically disclosed herein. All ranges disclosed herein are also to be considered to include the end points of the range, unless expressly stated otherwise. For example, a range of “between 5 and 10” or “5 to 10” or “5-10” should be considered to include the end points 5 and 10.

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified may be employed in the practice of the disclosure without resort to undue experimentation. All art-known functional equivalents, of any such methods, device elements, and materials are intended to be included in this disclosure.

The term “effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

An “effective amount,” “prophylactically effective amount,” or “therapeutically effective amount” may be first estimated either in accordance with cell culture assays or using animal models, typically mice, rats, guinea pigs, rabbits, dogs or pigs. An animal model can be used to determine an appropriate concentration range and route of administration. Such information can then be used to determine appropriate doses and routes of administration for humans. When calculating a human equivalent dose, a conversion table such as that provided in Guidance for Industry: Estimating the Maximum Safe Starting Dose in Initial Clinical Trials for Therapeutics in Adult Healthy Volunteers (U.S. Department of Health and Human Services, Food and Drug Administration, Center for Drug Evaluation and Research (CDER), July 2005) can be used. The person of ordinary skill in the art is aware of additional guidance that may also be used to develop human therapeutic dosages based on non-human data. An effective dose is generally 0.01 mg/kg to 2000 mg/kg of an active agent, preferably 0.05 mg/kg to 500 mg/kg of an active agent. An exact effective dose will depend on the severity of the disease, patient's general state of health, age, body weight and sex, nutrition, time and frequency of administration, combination(s) of medicines, response sensitivity and tolerance/response to administration and other factors that will be taken into account by a person skilled in the art when determining the dosage and route of administration for a particular patient based on his/her knowledge of the art. Such dose may be determined by conducting routine experiments and at the physician's discretion. Effective doses will also vary depending on the possibility of their combined use with other therapeutic procedures, such as the use of other agents.

As used herein, a “patient” and a “subject” are interchangeable terms and refer to a human patient/subject, a dog, a cat, a non-human primate, etc.

Unless defined otherwise, all technical and scientific terms used herein have the same meaning as commonly understood by one of ordinary skill in the art to which the disclosure pertains. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

COVID-19

The Sars-Cov-2 virus enters human cells by using its envelope homotrimeric spike glycoprotein (SPK or SP) to bind to its host's cellular receptor ACE-2. The binding between the glycoprotein and ACE-2 receptor leads to a fusion between viral and host cell membranes in preparation for the virus to enter the cell. Chen H. et al., (2020), medRxiv. doi: https://doi.org/10.1101/2020.02.15.20023457. After binding to ACE-2, the TMPRSS-2 adhesion proteins helps the spike protein complex to further invade the human body by cleaving the spike protein. There is an abundance of TMPRSS-2 proteins in locations where ACE-2 proteins are present. Urszula R. et al., (2020), Allergy, November; 75(11):2829-2845. doi: 10.1111/all.14429. Epub 2020 Aug. 24. The Sars-Cov-2 spike protein adheres to the TMPRSS2 proteins and are then cleaved at multiple sites, resulting in the spread of these virus proteins around the body. These cleavages also decrease the sensitivity of the antibodies produced by the immune system, rendering it harder to eliminate all the virus proteins. Ilona G. et al., (2011), Journal of virology, May; 85(9):4122-34. doi: 10.1128/JVI.02232-10. Epub 2011 Feb. 16. Therefore, there is a correlation between various adhesion proteins and ACE-2 receptors. Sars-Cov-2 spike proteins' invasion of the human body can be remediated by decreasing the amount of adhesion molecules inside the human body.

Apoptosis, Also known as programmed cell death, is Sars-Cov-2 spike protein's main mechanism of damaging the human body. Ren Y. et al., (2020), Cell Mol Immunology, volume 17 page 881-883. During apoptosis, outer mitochondrial membrane permeabilization is induced by cytotoxic stimuli to prepare for caspase activation. As a result of this permeabilization, cytochromes are released, resulting in activation of caspase 9, which cleaves substrates in the cells to induce apoptosis. Hung, R. W. Y., & Chow, A. W. (1997), Canadian Journal of Infectious Diseases, 8(2), 103-109. Thus, decrease in caspase 9 expression would reduce apoptosis.

Necrosis is another mechanism for the Sars-Cov-2 spike protein to damage the human body. David A. Schwartz, and Denise Morotti, Viruses, 2020 Nov. 15; 12(11):1308. doi: 10.3390/v12111308. Thus, reducing necrosis caused by Sars-cov-2 spike proteins will be another crucial step in the remediation of Covid-19 damage.

The level of Sars-Cov-2 spike proteins are directly proportional to the amount of cytokines in the human body. Gu T. et al., Front Immunol, 2021 Jan. 28; 11:621441. doi: 10.3389/fimmu.2020.621441. eCollection 2020. This increase in cytokine expression leads to a cytokine storm resulting in dysfunction of the immune system and increases the chance of immune cells being infected with covid-19. Id. While immune cells are less likely to be infected, the damages are much more severe compared to other cells. Thus, it is important to try and decrease the cytokine expression in the patient before covid-19 starts infecting immune cells in the human body. Ropa, J., et al., Stem Cell Rev Rep, 2021 February; 17(1):253-265. doi: 10.1007/s12015-020-10056-z. Epub 2020 Oct. 21.

CD26 are antibodies on the surface of T-cells. CD26 cleaves amino-terminal dipeptides from polypeptides in the penultimate position, leading to T-cell activation. CD26 is a key immunoregulatory factor in viral infections. As Sars-cov-2 virus particles enter the body, they target CD26, resulting in immune system dysfunction. As a result of Sars-cov-2 spike proteins, the proliferation of T-cells stops and the immune system will no longer be able to eliminate the viruses. Naveen Vankadari and Jacqueline A. Wilce, Emerg Microbes Infect. 2020 Mar. 17; 9(1):601-604. doi: 10.1080/22221751.2020.1739565. Thus, it is important to try and increase the CD26 expression to restore the immune system in efforts to combat Covid-19.

The possible application of Artemisinin (A1) and/or Artesunate (A2) and their derivatives as treatment for viruses has been considered. Sehailia Moussa and Smain Chemat (2020), Journal of biomolecular structure and dynamics, pages 1-11. A1 and A2, considered malaria and cancer treatment drugs, are also examined to have antiviral effects for other families of SPK such as 3CLpro corona virus. Id. Also, A1 and A2 inhibit monocyte cell adhesion. Wang, Y. et al., International Journal of Molecular Medicine, volume 37, Published online on: Apr. 26, 2016 https://doi.org/10.3892/ijmm.2016.2579, pp. 1567-1575.

Methods and Compositions

Once recovered from a Sar-cov-2 infection, patients tend not to be re-infected, as the patients have developed immunity to the virus. But many variants of Sar-cov-2 have been discovered from infected human patients since its emergence in 2019 as a human infectious agent. Some variants are different enough from the virus that infected a particular patient so that these variants can cause re-infection of that patient. Such variants likely are able to bypass the humoral immune response of the patient, at least enough to cause re-infection. The Omicron variant is likely one such variant.

In one aspect, this disclosure provides a method of treating Sar-cov-2 re-infection in a subject, wherein said re-infection is caused by a Sar-cov-2 virus variant that has bypassed the subject's humoral immune system, comprising administering an effective amount of a pharmaceutical composition comprising or consisting essential of artemisinin or artesunate to said subject. In some embodiments, the re-infection is caused by the Omicron variant of Sar-cov-2 virus. In some embodiments, an effective amount of a composition comprising or consisting essential of a derivative of artemisinin or artesunate can be administered instead.

First generation vaccines developed by Pfizer/BioNTech, Moderna, Johnson and Johnson, AstraZeneca, and others, cause bad side effects in some subjects. These side effects include flu-like symptoms. Such flu-like symptoms can be quite severe and debilitating.

In another aspect, this disclosure provides a method of reducing side effects in a subject suffering from side effects of Covid-19 vaccination, comprising administering an effective amount of a pharmaceutical composition comprising or consisting essential of artemisinin or artesunate to said subject. The vaccine is one that causes side effects in some subjects when administered to these subjects. In some embodiments, an effective amount of a composition comprising or consisting essential of a derivative of artemisinin or artesunate can be administered instead.

In another aspect, this disclosure provides a method of reducing apoptosis of immune cells or gut cells caused by a Sar-cov-2 spike protein in a subject, comprising administering an effective amount of a pharmaceutical composition comprising or consisting essential of artemisinin or artesunate to said subject. Apoptosis can be measured in the subject's cells by in vitro laboratory techniques known in the art. In some embodiments, an effective amount of a composition comprising or consisting essential of a derivative of artemisinin or artesunate can be administered instead.

In yet another aspect, this disclosure provides a method of reducing necrosis of immune cells or gut cells caused by a Sar-cov-2 spike protein in a subject, comprising administering an effective amount of a pharmaceutical composition comprising or consisting essential of artemisinin or artesunate to said subject. Necrosis can be measured in the subject's cells by in vitro laboratory techniques known in the art. In some embodiments, an effective amount of a composition comprising or consisting essential of a derivative of artemisinin or artesunate can be administered instead.

In another aspect, this disclosure provides a method of increasing CD26 level in immune cells in a subject infected with Sar-cov-2, comprising administering an effective amount of a pharmaceutical composition comprising or consisting essential of artemisinin or artesunate to said subject. CD26 levels in the subject's immune cells can be measured by in vitro laboratory techniques known in the art. In some embodiments, an effective amount of a composition comprising or consisting essential of a derivative of artemisinin or artesunate can be administered instead.

In another aspect, this disclosure provides a method of decreasing cell adhesion molecule level in immune cells in a subject infected with Sar-cov-2, comprising administering an effective amount of a pharmaceutical composition comprising or consisting essential of artemisinin or artesunate to said subject. Decreased cell adhesion molecule level in a subject's immune cells can be measured by in vitro laboratory techniques known in the art. In some embodiments, the cell adhesion molecule is TMPRSS-2. In some embodiments, an effective amount of a composition comprising or consisting essential of a derivative of artemisinin or artesunate can be administered instead.

In yet another aspect, this disclosure provides a method of making an anti-Sar-cov-2 compound comprising making and testing derivatives of artemisinin or artesunate for activity of one or more of:

increasing CD26 level in immune cells with lowered CD26 level caused by a Sar-cov-2 spike protein,
increasing adhesion protein level in immune cells with lowered adhesion protein level caused by a Sar-cov-2 spike protein, and
reducing apoptosis or necrosis of immune cells or gut cells caused by a Sar-cov-2 spike protein. In some embodiments, the adhesion protein is TMPRSS-2. Methods of measuring increasing CD26 level in immune cells is known in the art. Methods of measuring increased adhesion molecule level is known in the art. Methods of measuring apoptosis or necrosis of immune cells or gut cells are known in the art. See Examples. The derivative of artemisinin or artesunate made and tested by this method is one that is unknown.

A derivative of artemisinin or artesunate is one that differs from artemisinin or artesunate structurally but is structurally related to either artemisinin or artesunate. A person of ordinary skill in the art would be able to identify and make these derivatives using methods known in the art.

Known derivatives of artemisinin and artesunate exist. Artesunate is in fact a semi-synthetic derivative of artemisinin. This disclosure encompasses using the known derivatives in the disclosed methods, other than the methods of making an anti-Sar-cov-2 compound comprising making and testing derivatives of artemisinin or artesunate.

Formulations, Dose Levels, Regimens and Modes of Administration

The composition of a disclosed method can be administered to a subject in need thereof by any suitable mode of administration, any suitable frequency, and at any suitable, effective dosage.

The composition for use in a disclosed method may be in any suitable form and may be formulated for any suitable means of delivery. In some embodiments, the composition for use in a disclosed method is provided in a form suitable for oral administration, such as a tablet, pill, lozenge, capsule, liquid suspension, liquid solution, or any other conventional oral dosage form. The oral dosage forms may provide immediate release, delayed release, sustained release, or enteric release, and, if appropriate, comprise one or more coating. In some embodiments, the disclosed composition is provided in a form suitable for injection, such as subcutaneous, intramuscular, intravenous, intraperitoneal, or any other route of injection. In various embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

The composition for use in a disclosed method can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the composition for use in a disclosed method comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

Water may be used as a carrier and diluent in the composition. The use of other pharmaceutically acceptable solvents and diluents in addition to or instead of water is also acceptable. In certain embodiments, deuterium-depleted water is used as a diluent.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically. Suitable routes of administration include, for example, oral or parenteral administration, such as intravenous, intraperitoneal, intragastric as well as via drinking water. However, depending on a dosage form, the disclosed composition may be administered by other routes.

The composition for use in a disclosed method can be administered with any other agent in combination.

Further properties and characteristics of the disclosure will become more apparent hereinafter from the following detailed disclosure by way or illustration only with reference to the examples.

### EXAMPLES

For this invention to be better understood, the following examples are set forth. These examples are for purposes of illustration only and are not to be construed as limiting the scope of the invention in any manner.

### Example 1 Artemisinin and Artesunate Mitigate Recombinant Spike Protein of Sars-Cov-2 Induced Apoptosis, Necrosis, Caspase, Cytokine Expression, and Adhesion Molecule Expression in Immune Cells

A1 or A2 Decreases Adhesion Molecule Expression

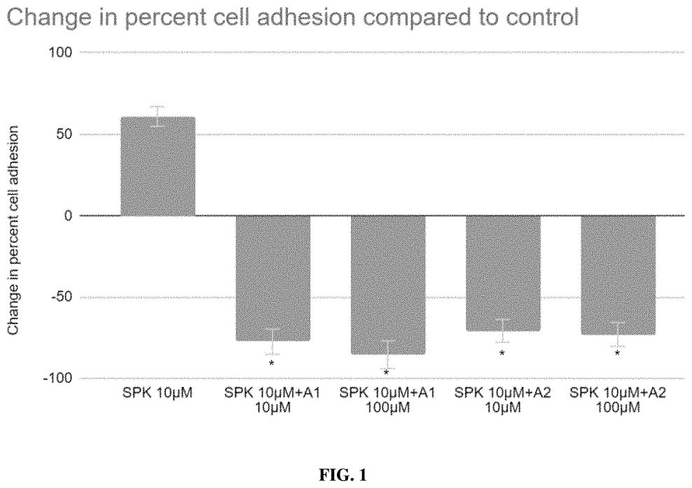

In the cell adhesion assay, SPK showed a 50 percent increase compared to the control. A1 or A2 showed a significant decrease in adhesion molecule expression when co-administered with SPK, with A1 decreasing the expression by around 70% and A2 by around 68%. These two chemicals also showed an increase in their effect as the concentration increases as A1 at 100 μM decreased the cell adhesion by about 73% and A2 at 100 μM decreased the cell adhesion by around 70%. Thus, A1 and A2 were both successful in decreasing the adhesion molecule expression and showed a dose dependent effect. See FIG. 1.

A1 or A2 Decreases LDH Expression Induced by SPK

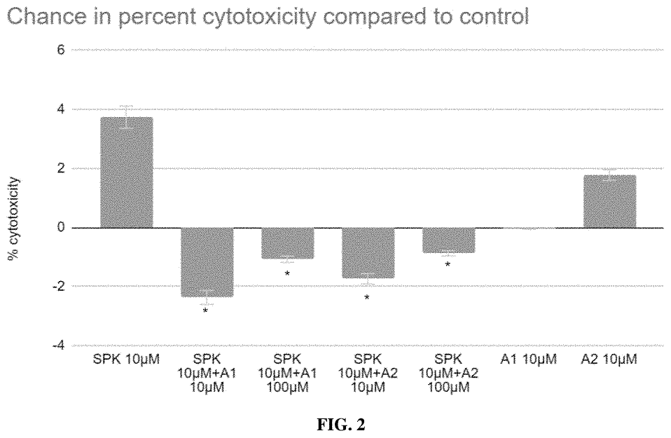

In the LDH assay, A1 showed a cytotoxicity decrease of around 2.4 percent and A2 showed a decrease around 0.8 percent at 10 μMs. The cytotoxicity is induced by SPK. See FIG. 2. However, as the concentrations of A1 and A2 increased, the effectiveness decreased. This could be a result of the chemicals being too toxic at a higher concentration and resulting in harming the U937 cells. U937 is a pro-monocytic, human myeloid leukemia cell line.

A1 or A2 Decreases Caspase Apoptosis Caused by SPK

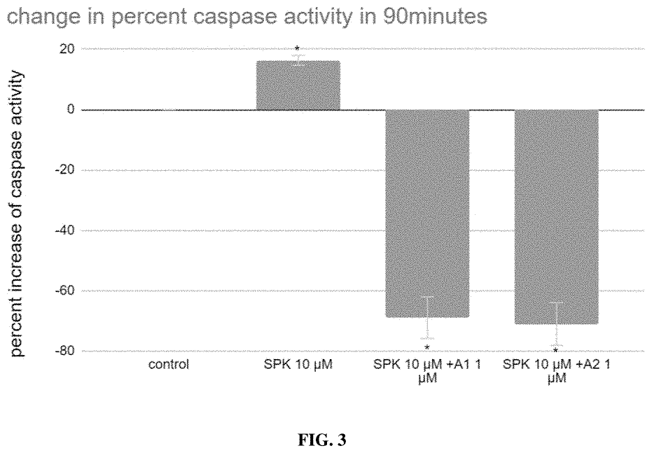

In the caspase assay, SPK is shown to increase the percent caspase activity by about 17 percent when compared to the control. However, A1 or A2 is able to decrease the caspase activity that was increased by SPK by around 68 and 70 percent respectively. As caspase activity is directly proportional to apoptosis. A1 or A2 are shown to remediate the apoptotic effect of SPK on U937 cells. See FIG. 3.

A1 Binding Effect on CD26

In molecular docking experiments, A1 showed a relatively high binding affinity to CD26, ranging from −6.9 to −7.8. These values indicate that A1 has a high binding affinity for CD26 and thus have a strong effect in increasing CD26 expression in the human body. Table 1.

TABLE 1
A1 Binding Affinity for CD26

| | Binding | | |
|---|---|---|---|
| Ligand | Affinity | msd/ub | msd/lb |
| 1j2e_tcicactvs000eL | −7.8 | 0 | 0 |
| 1j2e_tcicactvs000eL | −7.7 | 26.953 | 24.867 |
| 1j2e_tcicactvs000eL | −7.5 | 37.158 | 34.993 |
| 1j2e_tcicactvs000eL | −7.5 | 3.124 | 1.984 |
| 1j2e_tcicactvs000eL | −7.4 | 52.18 | 50.048 |
| 1j2e_tcicactvs000eL | −7.3 | 51.982 | 49.793 |
| 1j2e_tcicactvs000eL | −7.1 | 29.193 | 26.918 |
| 1j2e_tcicactvs000eL | −7 | 34.793 | 33.598 |
| 1j2e_tcicactvs000eL | −6.9 | 37.192 | 35.42 |

A1 or A2 Remediates SPK Necrosis to the Human Body

In the LDH assay, A1 or A2 reversed the cytotoxic effect of SPK from a positive value to a negative value. Cytotoxicity measures the overall damage a chemical has for the human body. Positive cytotoxicity levels indicate that the chemical deals damage to the human body when a negative value indicates the chemicals have a protective effect. The results indicate that when A1 or A2 is combined with SPK, SPK's damage to the human body is decreased in all aspects. A lower LDH expression also indicates a decrease in the necrosis which contributes to the decrease in overall damage.

A1 or A2 Remediating SPK Apoptosis to the Human Body

In the caspase assay, A1 or A2 was shown to be able to decrease SPK caused apoptosis significantly. This shows that A1 and A2 are able to significantly remediate the damages SPK does to human immune cells. In addition, with more immune cells surviving, the human immune system would become much more effective in fighting against covid-19.

A1 or A2 Decreasing Adhesion Molecule Expression in the Human Body

In the cell adhesion assay, A1 or A2 significantly decreased the adhesion molecule expression in the U937 cells. With lowered expression of adhesion molecules such as TMPRSS 2, it will be much harder for SPK to enter the human immune system and cause cytokine storms since there will be no more adhesion molecules to cleave the SPK.

A1 has High Binding Affinity to CD26

In the molecular docking assay, A1 showed a very high binding affinity to CD26, suggesting that A1 has a strong effect on CD26. With higher levels of CD26, it will be harder for the Sars-Cov-2 spike proteins to enter the immune system since more T-cells will be active to kill these viruses. Thus, if A1 is used on a patient, it not only decreases covid-19's damage but also prevents further infection to the human immune system.

Materials and Methods

Cell Culture

The cells used in this example originated from the cell line U937 cells and were purchased from ATCC.

LDH Assay

10 μls of chemicals provided by ATCC were added into a 96-well plate with the treatment plan of control, control, control, SPK 10 μM, A1 10 μM, A1 100 μM, A2 10 μM, A2 100 μM, SPK+A1 10 μM, SPK+A1 100 μM, SPK+A2 10 μM, and SPK+A2 100 μM. After incubation for 48 hours, 30 μls of media from each well were removed and added into another 96 well plate labeled as released LDH. 30 microliters of LDH substrate mix were added to the released LDH plate to measure the released LDH by the cells from the treatments. This plate was left at room temperature and away from direct light for 30 minutes. After 30 minutes, 30 μls of LDH stop solution was added to each well to terminate the assay and prevent further color changes. The plate was placed in a microplate reader and optical density (OD) was determined for each sample at 490 nm. Another reading at 655 nm was performed to determine if there was any background interference with the reading. Then, 10 μls of LDH lysis buffer were added to the original plate containing the cells to lyse all the cells and to measure a total or maximum LDH for each sample. Another empty 96 well plate was then labeled total LDH. 30 μls of the cell lysate and media were transferred from the original plate containing the lysed cells into the empty 96 well plate labeled as total LDH. 30 μls of LDH substrate mix were added to each sample to measure the total LDH. The plate was left at room temperature for 30 minutes away from direct light. After the 30 minutes, 30 μls of LDH stop solution were added to each well to terminate the experiment. The OD of the total LDH plate was measured following the same measurement steps for the released LDH. The data collected from the 490 nm wavelength was subtracted by the data collected from the 655 nm wavelength to find the corrected OD for each sample's released and total LDH. The percent cytotoxicity was later calculated by the equation: (Sample OD released LDH−control OD released LDH)/(Sample OD total LDH−control OD released LDH)*100 and T-tests were applied to each group to measure significance for percent cytotoxicity.

Adhesion Assay

For the adhesion assay, 10 μls of each chemical were added following the treatment plan of control, control, control, SPK 10 μM, A1 10 μM, A1 100 μM, A2 10 μM, A2 100 μM, SPK+A1 10 μM, SPK+A1 100 μM, SPK+A2 10 μM, and SPK+A2 100 μM. Blocking buffers were added into each well and washed out with a washing buffer after 60 minutes. After 15 minutes the washing buffer was replaced with dye and washed out with water after another 15 minutes. The plate was then be read at 555 nm and pictures will be taken using a microscope. The amount of cells attached will be measured in ImageJ from changing the picture type to 8 bit, subtracting the background, adjusting threshold, and Plugins ICTN.

Caspase 9 Assay

The cells were treated in a 6-well plate with the treatment plan of control, A1 10 μM, A2 10 μM, SPK10 μM, A1+SPK10 μM, and A2+SPK10 μM for 24 hours and collected in a 1.5 ml tube. Then, Colo cells were lysed by freezing and thawing 4 times. Then, 50 μl of caspase assay buffer will be added to every well of an empty 96-well plate. Also, 50 μl of lysis buffer will be added to the blank wells and 45 μl added to the treatment and control wells. 5 μl of lysed cells will be added to each treatment well with 5 μl of pNA Substrate Solution indeed to every well. The plate will then be taken for a reading at zero time point using a microplate reader at 405 nm. Afterwards, the plate will be covered and incubated at 37 degrees Celsius. Every 30 minutes the plate will be taken out and read with a microplate reader until all the values are significantly different from the zero point. The data from the Caspase Assay will be analyzed by calculating the change in optical density per minute (ΔOD). The ΔOD is obtained through subtracting the sample's start OD from the sample's final OD to determine the overall change in absorbance. The ΔOD/minutes is calculated by taking the ΔOD and dividing by the overall time in minutes. The ΔOD per minute of each sample is compared to the control by a percent change to determine the activity of caspase and apoptosis. The OD of the blank samples are subtracted from the OD of the treated sample to normalize the data and ensure minimal interference.

Molecular Docking

Molecular docking is performed through the computer program pyrx. The molecular structure of the Sars-COV-2 Spike protein is retrieved from the protein data bank. The molecular structure of A1 and A2 were found online and converted to a pdb file to perform molecular docking using openbabel. The pdb files of the chemicals and the spike protein were put into the software pyrx and a vina test was performed to determine binding affinity.

### Example 2 Artemisinin and Artesunate Mitigate Recombinant Spike Protein of Sars-Cov-2 Induced Apoptosis and Necrosis in Gut Endothelial Cells

Recombinant SPK Induction of Apoptosis and Necrosis of Colo 320 Cells

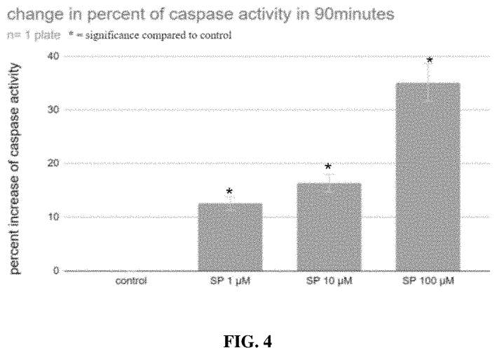

FIG. 4 shows that SPK is able to induce caspase apoptosis in colo 320 cells, a human colon carcinoma cell line, at a dose dependent manner, but the effect is only significant at higher concentrations.

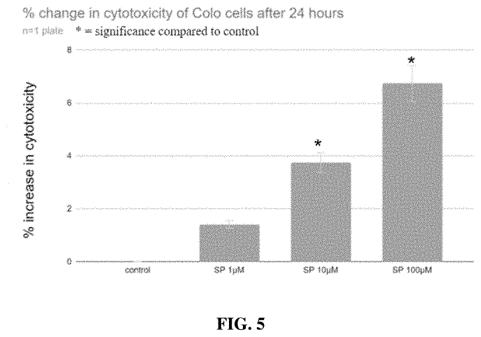

FIG. 5 shows that SPK induces increased level of cytotoxicity in a dose dependent matter in colo cells which indicates that SPK also damages the human body through necrosis.

A1 or A2 Mitigation of SPK Induced Apoptosis

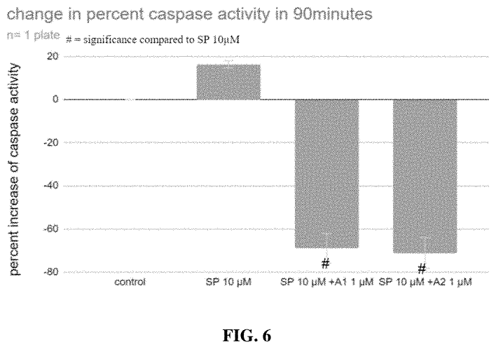

FIG. 6 shows that A1 or A2 is able to remediate the increase in caspase apoptosis to a negative level which shows that the two chemicals are able to improve cell proliferation.

A1 and A2 Mitigation of SPK Induced Necrosis

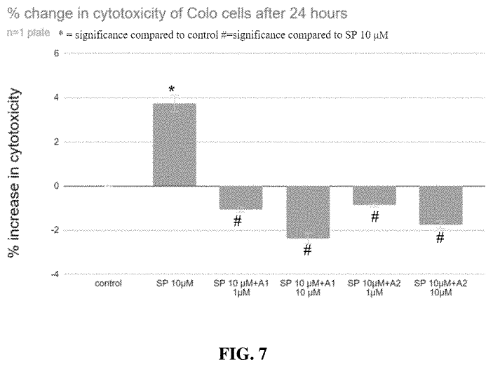

Based on FIG. 7, SPK had a positive cytotoxicity which means it induces necrosis to the human body, but this damage is remediated by A1 or A2, which changed the cytotoxicity to a negative value that protects the cells.

Molecular Docking Indication of A1 or A2 Binding with SPK

In Table 2, A1 showed a high negative binding affinity of SPK, showing strong bind to the spike protein and infers that it will interfere with the binding of SPK to Ace-2 receptors.

TABLE 2
Binding affinity of A1 for the
Sars-Cov-2 spike protein

| | Binding |
|---|---|
| | Affinity |
| Ligand | (kcal/mol) |
| 6lzg_B_artemisinin | −9.1 |
| 6lzg_B_artemisinin | −8.7 |
| 6lzg_B_artemisinin | −8.5 |
| 6lzg_B_artemisinin | −8.4 |
| 6lzg_B_artemisinin | −8.3 |
| 6lzg_B_artemisinin | −8.1 |
| 6lzg_B_artemisinin | −8.1 |
| 6lzg_B_artemisinin | −8.1 |
| 6lzg_B_artemisinin | −7.9 |

Materials and Methods

Cell Culture

The cells used in this example originated from the cell line Colo 320 and were purchased from ATCC; previous studies show these cells express ACE-2 receptor. Chen H. et al., (2020), medRxiv. doi: https://doi.org/10.1101/2020.02.15.20023457.

MTT Assay

Protocols from vendors are followed with the treatment plan of control, A1 1 μM, A1 10 μM, A2 1 μM, A2 10 μM, SP 1 μM, SP 10 μM, SP+A1 1 μM, SP+A2 1 μM, SP+A1 10 μM, SP+A2 10 μM

LDH Assay

Protocols from vendors are followed with the treatment plan of control, A1 1 μM, A1 10 μM, A2 1 μM, A2 10 μM, SP 1 μM, SP 10 μM, SP+A1 1 μM, SP+A2 1 μM, SP+A1 10 μM, SP+A2 10 μM.

Caspase 9 Assay

Protocols from vendors are followed with the treatment plan of control, A1 1 μM, A1 10 μM, A2 1 μM, A2 10 μM, SP 1 μM, SP 10 μM, SP+A1 1 μM, SP+A2 1 μM, SP+A1 10 μM, SP+A2 10 μM.

Molecular Docking

Molecular docking is performed through the computer program pyrx. The molecular structure of A1, A2 and SP were put into the software pyrx and a vina test was performed to determine binding affinity.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

A number of embodiments of the disclosure have been described. The specific embodiments provided herein are examples of useful embodiments of the invention and it will be apparent to one skilled in the art that the disclosure can be carried out using a large number of variations of the devices, device components, methods steps set forth in the present description. As will be obvious to one of skill in the art, methods and devices useful for the present methods may include a large number of optional composition and processing elements and steps.

1-2. (canceled)

3. A method of reducing side effects in a subject suffering from side effects of Covid-19 vaccination, comprising administering an effective amount of a pharmaceutical composition comprising artemisinin or artesunate to said subject.

4-8. (canceled)

9. (canceled)
